(19) United States
(12) Patent Application Publication
(10) Pub. No.: US 2022/0228158 A1
(43) Pub. Date: Jul. 21, 2022

(54) INDIGO PLANT POPULATIONS WITH HIGH INDICAN CONTENT

(71) Applicant: Stony Creek Colors, Inc., Springfield, TN (US)

(72) Inventor: Shawn Genung, Springfield, TN (US)

(21) Appl. No.: 17/578,807

(22) Filed: Jan. 19, 2022

Related U.S. Application Data
(60) Provisional application No. 63/199,708, filed on Jan. 19, 2021.

(51) Int. Cl. C12N 15/82; A61K 36/48; A01H 1/02; A01H 1/04

(52) U.S. Cl. CPC C12N 15/825; A01H 1/04; A01H 1/02; A61K 36/48

(57) ABSTRACT

According to the invention, there is provided a novel population of indigo plants (Indigofera suffruticosa) producing a higher than average level of indican. This invention thus relates to an assemblage of indigo seeds of the population, to the indigo plants of population, and to methods for producing indigo varieties with high indican content derived from the indigo plants of the population.

### CROSS-REFERENCE TO RELATED APPLICATION

This application claims priority under 35 U.S.C. § 119 to Provisional Application U.S. Ser. No. 63/199,708, filed on Jan. 19, 2021, which is herein incorporated by reference in its entirety including without limitation, the specification, claims, and abstract, as well as any figures, tables, or examples thereof.

### GRANT REFERENCE

This invention was made with government support under grant numbers 1831949 and 1622860 awarded by the National Science Foundation. The government has certain rights in this invention.

### FIELD OF THE INVENTION

This invention is in the field of plant breeding, specifically relating to Indigofera suffruticosa populations producing a higher than average level of indican.

### BACKGROUND TO THE INVENTION

Indigo has been used to dye fabric with indigo blue since before recorded history. The sap which oozes from the plant when bruised was applied to fabric by ancient Egyptians, Greeks, and Romans. Indigo has been used in India to dye fabric for at least 4,000 years by methods which are practically identical to methods employed today. Indigo was introduced in Europe in large quantities by the Dutch East India Company in the early 17th century.

Leaves of indigo plants contain indican, the precursor of indigo dye. While indigo plant leaves continue to be grown for dye, most indigo dye used in commercial dyeing today is synthetic. The production of synthetic indigo dye requires the use of toxic materials which can be highly polluting. Thus, there has been a renewed interest in the use of natural indigo dye to address the environmental concerns associated with synthetic production.

The present invention relates to a population of indigo plants (Indigofera suffruticosa) selected for high indican content and varieties derived therefrom for use in the production of natural indigo dye.

### SUMMARY OF THE INVENTION

A population of Indigofera suffruticosa germplasm has been bred and selected to achieve a higher than average amount of indigo dye per plant. The population has also been selected and adapted for the Southeastern United States growing region. The population provides a unique source of germplasm for traits which can be used to develop varieties of indigo plants for dye extraction.

Thus, the invention encompasses an assemblage of indigo seeds of the population, the indigo plants of the population, plant parts of the indigo plants of the population (including leaves, seeds, gametes), methods of producing an assemblage of indigo seeds from the population, and methods for producing a population of progeny indigo plants by crossing an indigo plant of the population with itself or another indigo plant. The invention also relates to methods for producing other indigo plants derived from the indigo plants of the population and to indigo plants, parts thereof and seeds derived by the use of those methods. The present invention further relates to indigo seeds and plants (and parts thereof including leaves) produced by crossing an indigo plant of the population with itself or with another indigo plant (e.g., an F₁ hybrid seed or plant).

In another aspect, the present invention provides regenerable cells for use in tissue culture of an indigo plant of the population. In embodiments, the tissue culture is capable of regenerating plants having all or essentially all of the physiological and morphological characteristics of the foregoing population and/or of regenerating plants having the same or substantially the same genotype as the foregoing population. In exemplary embodiments, the regenerable cells in such tissue cultures are meristematic cells, cotyledons, hypocotyl, leaves, pollen, embryos, roots, root tips, anthers, pistils, ovules, shoots, stems, petiole, flowers, and/or seeds as well as callus and/or protoplasts derived from any of the foregoing. Still further, the present invention provides a population of indigo plants regenerated from the tissue cultures of the invention.

As a further aspect, the invention provides a method of producing an assemblage of indigo seeds, the method comprising selecting an indigo plant of the population with high indican content, and crossing the selected indigo plant with itself or a second indigo plant. The indigo plant of the population can be the female and/or male parent. In embodiments, the crossing comprises open pollination. Optionally, the method further comprises collecting the seeds. The invention also provides seeds produced by this method and plants produced by growing the seeds.

The invention further provides a method of producing a population of progeny indigo plants, the method comprising selecting an indigo plant of the population with high indican content, crossing the selected indigo plant with itself or a second indigo plant to produce at least a first progeny plant, which may optionally be a selfed plant or an F₁ hybrid. The indigo plant of the population can be the female and/or male parent.

Another aspect of the invention provides methods for producing hybrids and other indigo plants derived from the indigo plants of the population. Indigo plants derived by the use of those methods are also part of the invention as well as plant parts, seeds, gametes and tissue culture from such hybrid or derived indigo plants.

In representative embodiments, indigo plants derived from the population comprise cells comprising at least one set of chromosomes derived from the indigo plants of the population. In embodiments, an indigo plant or population of indigo plants derived from the indigo plants of the population comprise, on average, at least about 6.25%, 12.5%, 25%, 30%, 35%, 40%, 45%, 50%, 55%, 60%, 65%, 70%, 80%, 85%, 90%, 95%, 96%, 97%, 98% or 99% of its alleles from the indigo plants of the population, e.g., at least about 6.25%, 12.5%, 25%, 30%, 35%, 40%, 45%, 50%, 55%, 60%, 65%, 70%, 80%, 85%, 90%, 95%, 96%, 97%, 98% or 99% of the genetic complement of the indigo plants of the population. In embodiments, the indigo plants derived from the indigo plants of the population are one, two, three, four, five or more breeding crosses removed from the indigo plants of the population.

In embodiments, hybrid or derived plants from the indigo plants of the population comprise a desired added trait(s). In representative embodiments, indigo plants derived from the indigo plants of the population comprise some or all of the morphological and physiological characteristics of the population (e.g., as described herein, in particular, high indican content). In embodiments, the indigo plants derived from the indigo plants of the population comprise essentially all of the morphological and physiological characteristics of the population (e.g., as described herein, in particular, high indican content), with the addition of a desired added trait(s).

The invention also relates to methods for producing indigo plants comprising in their genetic material one or more transgenes and to the transgenic indigo plants produced by those methods (and progeny indigo plants comprising the transgene). Also provided are plant parts, seeds, and tissue culture from such transgenic indigo plants, optionally wherein one or more cells in the plant parts, seeds, or tissue culture comprise the transgene. The transgene can be introduced via plant transformation and/or breeding techniques.

Another aspect of the invention provides methods of editing the genome of the indigo plants including by use of the Clustered Regularly Interspaced Short Palindromic Repeats (CRISPR)/Cas system, Transcription Activator-Like Effector Nucleases (TALENs), or Zinc Finger Nucleases (ZFN), and the edited indigo plants produced by those methods. Also provided are plant parts, seeds, and tissue culture from such edited indigo plants.

In another aspect, the present invention provides for single locus converted indigo plants. Plant parts, seeds, and tissue culture from such single locus converted plants are also contemplated by the present invention. The single locus may be a dominant or recessive allele. In representative embodiments, the single locus confers such traits as male sterility, herbicide resistance, pest resistance (e.g., insect and/or nematode resistance), disease resistance (e.g., for bacterial, fungal and/or viral disease), male fertility, improved salt tolerance, industrial usage, or any combination thereof. The single locus may be a naturally occurring gene, a genome edited locus, a mutated locus (e.g., chemically or radiation induced), or a transgene introduced through genetic engineering techniques.

The invention further provides methods for developing indigo plants in an indigo plant breeding program using plant breeding techniques including, for example, recurrent selection, backcrossing, pedigree breeding, double haploid techniques, restriction fragment length polymorphism enhanced selection, genetic marker enhanced selection, genome editing, and/or transformation. Seeds, indigo plants, and parts thereof, produced by such breeding methods are also part of the invention.

The invention also provides methods of multiplication or propagation of indigo plants of the invention, which can be accomplished using any method known in the art, for example, via vegetative propagation and/or seed.

The invention further provides a method of producing indican or indigo dye from the population comprising (a) obtaining a population of indigo plants of the invention, optionally wherein the plants have been cultivated to maturity, and (b) collecting the indigo plants or parts thereof (e.g., leaves) from the plants. In embodiments, obtaining the indigo plants comprises growing the plants.

Additional aspects of the invention include harvested products and processed products from the population of indigo plants of the invention. A harvested product can be whole plants or any plant part, as described herein. Thus, in some embodiments, a non-limiting example of a harvested product includes leaves. In representative embodiments, a processed product includes indican or indigo dye that is extracted, purified or isolated from the population of indigo plants of the invention. Thus, the invention also provides a method of producing a processed product from the population of indigo plants of the invention, the method comprising (a) obtaining leaves of the population of indigo plants of the invention; and (b) processing the leaves to produce a processed product. In embodiments, the processing comprises extracting, purifying, and/or isolating.

As another aspect, the invention provides leaves and/or seeds of the population of indigo plants of the invention and a processed product from leaves and/or seed of the inventive population of indigo plants.

As yet a further aspect, the invention provides a method for producing seeds of an indigo plant derived from the indigo plants of the population, the method comprising: (a) selecting an indigo plant of the population with high indican content; (b) crossing the selected indigo plant with a second indigo plant; (c) allowing seed to form; (d) growing a plant from the seed of step (c) to produce an indigo plant derived from the indigo plants of the population; (e) selfing the plant of step (d) or crossing it to a second indigo plant to form additional indigo seed derived from the indigo plants of the population; and (f) optionally repeating steps (d) and (e) one or more times (e.g., one, two, one to three, one to five, one to six, one to seven, one to ten, three to five, three to six, three to seven, three to eight or three to ten times) to generate further derived indigo seed from the indigo plants of the population, wherein in step (d) a plant is grown from the additional indigo seed of step (e) in place of growing a plant from the seed of step (c). As another option, in embodiments, the method comprises collecting the indigo seeds. The invention also provides seeds produced by these methods and plants derived from the indigo plants of the population produced by growing the seeds.

As another aspect, the invention is also directed to a method of producing indigo leaves comprising obtaining indigo plants of the population according to the invention and harvesting leaves from the plants. In embodiments, obtaining indigo plants of the invention comprises growing the plants to produce leaves. In one embodiment, the method further comprises processing the leaves to obtain indican or indigo dye.

Still further, as another aspect, the invention provides a method of vegetatively propagating an indigo plant of the population. In a non-limiting example, the method comprises: (a) selecting an indigo plant of the population with high indican content; (b) collecting tissue capable of being propagated from the selected indigo plant; (c) cultivating the tissue to obtain proliferated shoots; and (d) rooting the proliferated shoots to obtain rooted plantlets. Optionally, the invention further comprises growing plants from the rooted plantlets. The invention also encompasses the plantlets and plants produced by these methods.

As an additional aspect, the invention provides a method of introducing a desired added trait into an indigo plant of the population, the method comprising: (a) selecting an indigo plant of the population with high indican content; (b) crossing a first selected indigo plant of the population with a second indigo plant that comprises a desired trait to produce F₁progeny; (c) selecting an F₁ progeny that comprises the desired trait; (d) crossing the selected F₁ progeny with an indigo plant of the population to produce backcross progeny; and (e) selecting backcross progeny comprising the desired trait to produce a plant derived from the indigo plants of the population comprising a desired trait. In embodiments, the selected progeny has one or more of the characteristics of the population (e.g., as described herein, in particular, high indican content). In embodiments, the selected progeny comprises all or essentially all the morphological and physiological characteristics of the population. Optionally, the method further comprises: (f) repeating steps (d) and (e) one or more times (e.g., one, two, one to three, one to five, one to six, one to seven, one to ten, three to five, three to six, three to seven, three to eight or three to ten times) to produce a plant derived from the indigo plants of the population comprising the desired trait, wherein in step (d) the selected backcross progeny produced in step (e) is used in place of the selected F₁ progeny of step (c).

In representative embodiments, the invention also provides a method of producing an indigo plant of the population comprising a desired added trait, the method comprising selecting an indigo plant of the population with high indican content, and introducing a transgene conferring the desired trait into the selected indigo plant. The transgene can be introduced by transformation methods (e.g., genetic engineering) or breeding techniques. In embodiments, the plant comprising the transgene has one or more of the morphological and physiological characteristics of the population (e.g., as described herein, in particular, high indican content). In embodiments, the plant comprising the transgene comprises all or essentially all of the morphological and physiological characteristics of the population. In some embodiments, the desired added trait is introduced by genome editing.

The invention also provides indigo plants produced by the methods of the invention or a selfed progeny thereof, wherein the indigo plants have the desired added trait as well as seeds from such indigo plants.

According to the foregoing methods, the desired added trait can be any suitable trait known in the art including, for example, male sterility, male fertility, herbicide resistance, insect or pest (e.g., insect and/or nematode) resistance, disease resistance (e.g., for bacterial, fungal and/or viral disease), improved salt tolerance, industrial usage, or any combination thereof. In representative embodiments, a transgene conferring herbicide resistance confers resistance to glyphosate, sulfonylurea, imidazolinone, dicamba, glufosinate, phenoxy proprionic acid, L-phosphinothricin, cyclohexone, cyclohexanedione, triazine, benzonitrile, or any combination thereof. In representative embodiments, a transgene conferring pest resistance (e.g., insect and/or nematode resistance) encodes a Bacillus thuringiensis endotoxin.

In addition to the exemplary aspects and embodiments described above, the invention is described in more detail in the description of the invention set forth below.

### DESCRIPTION OF THE FIGURES

FIG. 1 shows the distribution of dye content of 926 plants at Seed Farm A in 2019.

FIG. 2 shows the distribution of dye content of 4531 plants at Seed Farm F in 2019.

FIG. 3 shows the distribution of dye content of 2936 plants in 2020.

### DETAILED DESCRIPTION OF THE INVENTION

The present invention is based, in part, on the development of a novel population of Indigofera suffruticosa plants.

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features and embodiments of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

All publications, patent applications, patents, and other references mentioned herein are incorporated by reference in their entirety.

### Definitions

In the description that follows, a number of terms are used. In order to provide a clear and consistent understanding of the specification and claims, including the scope to be given such terms, the following definitions are provided:

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

“Allele”. An allele is any of one or more alternative forms of a gene, all of which relate to a trait or characteristic. In a diploid cell or organism, the two alleles of a given gene occupy corresponding loci on a pair of homologous chromosomes.

“Backcrossing”. Backcrossing is a process in which a breeder repeatedly crosses hybrid progeny back to one of the parents, for example, a first generation hybrid F₁ with one of the parental genotype of the F₁ hybrid.

“Cell”. Cell as used herein includes a plant cell, whether isolated, in tissue culture or incorporated in a plant or plant part. The cell can be a cell, such as a somatic cell, of the variety having the same set of chromosomes as the cells of the deposited seed, or, if the cell contains a locus conversion or transgene, otherwise having the same or essentially the same set of chromosomes as the cells of the deposited seed.

“Cotyledon”. One of the first leaves of the embryo of a seed plant; typically one or more in monocotyledons, two in dicotyledons, and two or more in gymnosperms.

“Double haploid line”. A stable inbred line achieved by doubling the chromosomes of a haploid line, e.g., from anther culture. For example, some pollen grains (haploid) cultivated under specific conditions develop plantlets containing 1 n chromosomes. The chromosomes in these plantlets are then induced to “double” (e.g., using chemical means) resulting in cells containing 2n chromosomes. The progeny of these plantlets are termed “double haploid” and are essentially non-segregating (e.g., are stable). The term “double haploid” is used interchangeably herein with “dihaploid.”

“Elite line”. Elite line means any line that has resulted from breeding and selection for superior agronomic performance. An “elite population” is an assortment of elite individuals or lines that can be used to represent the state of the art in terms of agronomically superior genotypes of a given crop species. Similarly, an “elite germplasm” or elite strain of germplasm is an agronomically superior germplasm.

“Essentially all the physiological and morphological characteristics”. In embodiments, a plant having “essentially all of the physiological and morphological characteristics” (and similar phrases) means a plant having the physiological and morphological characteristics of the population, except for the characteristics derived from the converted locus/loci (e.g., a single converted locus), for example, introduced via backcrossing, a modified gene(s) resulting from genome editing techniques, an introduced transgene (i.e., introduced via genetic transformation techniques) or mutation, when both plants are grown under the same environmental conditions. In embodiments, a plant having “essentially all of the physiological and morphological characteristics” means a plant having all of the characteristics of the reference plant with the exception of five or fewer traits, 4 or fewer traits, 3 or fewer traits, 2 or fewer traits, or one trait. According to representative embodiments, a plant having “essentially all of the physiological and morphological characteristics” (and similar phrases) has high indican content.

“F_{#}”. The “F” symbol denotes the filial generation, and the # is the generation number, such as F₁, F₂, F₃, etc.

“F₁ Hybrid”. The first-generation progeny of the cross of two nonisogenic plants.

“Gene”. As used herein, “gene” refers to a segment of nucleic acid comprising an open reading frame. A gene can be introduced into a genome of a species, whether from a different species or from the same species, using transformation or various breeding methods.

“Gene silencing”. The interruption or suppression of the expression of a gene at the level of transcription or translation.

“Genetic complement”. As used herein, a “genetic complement” refers to the total genetic make-up of the plant.

“Genetically modified”. Describes an organism that has received genetic material from another organism, or had its genetic material modified, resulting in a change in one or more of its phenotypic characteristics. Methods used to modify, introduce or delete the genetic material may include mutation breeding, genome editing, RNA interference, gene silencing, backcross conversion, genetic transformation, single and multiple gene conversion, and/or direct gene transfer.

“Genome editing”. A type of genetic engineering in which DNA is inserted, replaced, modified or removed from a genome using artificially engineered nucleases or other targeted changes using homologous recombination. Examples include but are not limited to use of zinc finger nucleases (ZFNs), TAL effector nucleases (TALENs), meganucleases, CRISPR/Cas9, and other CRISPR related technologies. (Ma et. al., Molecular Plant, 9:961-974 (2016); Belhaj et. al., Current Opinion in Biotechnology, 32:76-84 (2015)).

“Genotype”. Refers to the genetic constitution of a cell or organism.

“Inbred line”. As used herein, the phrase “inbred line” refers to a genetically homozygous or nearly homozygous population. An inbred line, for example, can be derived through several cycles of sib crossing and/or selfing and/or via double haploid production. In some embodiments, inbred lines breed true for one or more traits of interest. An “inbred plant” or “inbred progeny” is an individual sampled from an inbred line.

“Linkage”. Refers to a phenomenon wherein alleles on the same chromosome tend to segregate together more often than expected by chance if their transmission was independent.

“Linkage disequilibrium”. Refers to a phenomenon wherein alleles tend to remain together in linkage groups when segregating from parents to offspring, with a greater frequency than expected from their individual frequencies.

“Locus”. A defined segment of DNA.

“Locus conversion”. A locus conversion (also called a “trait conversion” or “gene conversion”) refers to a plant or plants within a variety or line that have been modified in a manner that retains the overall genetics of the variety and further comprises one or more loci with a specific desired trait, such as but not limited to male sterility, insect or pest control, disease control or herbicide tolerance. Examples of single locus conversions include mutant genes, transgenes and native traits finely mapped to a single locus. One or more locus conversion traits may be introduced into a single cultivar.

“Plant.” As used herein, the term “plant” includes plant cells, plant protoplasts, plant cell tissue cultures from which plants can be regenerated, plant calli, plant clumps, and plant cells that are intact in plants or parts of plants, such as leaves, pollen, embryos, cotyledons, hypocotyl, roots, root tips, anthers, pistils, flowers, ovules, seeds, stems, and the like.

“Plant Cell”. As used herein, the term “plant cell” includes plant cells whether isolated, in tissue culture or incorporated in a plant or plant part.

“Plant adaptability”. A plant having a good plant adaptability means a plant that will perform well in different growing conditions and seasons.

“Plant material”. The terms “plant material” and “material obtainable from a plant” are used interchangeably herein and refer to any plant material obtainable from a plant including without limitation, leaves, stems, roots, flowers or flower parts, fruits, pollen, ovules, zygotes, seeds, cuttings, cell or tissue cultures, or any other part or product of the plant.

“Plant part”. As used herein, a “plant part” includes any part, organ, tissue or cell of a plant including without limitation an embryo, meristem, leaf, pollen, cotyledon, hypocotyl, root, root tip, anther, flower, flower bud, pistil, ovule, seed, shoot, stem, stalk, petiole, and/or a rootstock including callus and protoplasts derived from any of the foregoing.

“Regeneration”. Regeneration refers to the development of a plant from tissue culture.

“Single locus converted”. A single locus converted or conversion plant refers to a plant that is developed by plant breeding techniques (e.g., backcrossing), genome editing techniques, genetic transformation techniques and/or mutation techniques wherein essentially all of the desired morphological and physiological characteristics of a line are recovered in addition to the single locus introduced into the line via the plant breeding, genome editing, genetic transformation, or mutation techniques.

“Substantially equivalent characteristic”. A characteristic that, when compared, does not show a statistically significant difference (e.g., p=0.05) from the mean.

“Transgene”. A nucleic acid of interest that can be introduced into the genome of a plant by genetic engineering techniques (e.g., transformation) or breeding. The transgene can be from the same or a different species. If from the same species, the transgene can be an additional copy of a native coding sequence or can present the native sequence in a form or context (e.g., different genomic location and/or in operable association with exogenous regulatory elements such as a promoter) than is found in the native state. The transgene can comprise an open reading frame encoding a polypeptide or can encode a functional non-translated RNA (e.g., RNAi).

### Indican and Indigo Dye

Indigo plants contain the glycoside indican, which is colorless. The hydrolysis of indican yields β-D-glucose and indoxyl. Reaction of indoxyl with a mild oxidizing agent, such as atmospheric oxygen, yields the indigo dye which is blue in color.

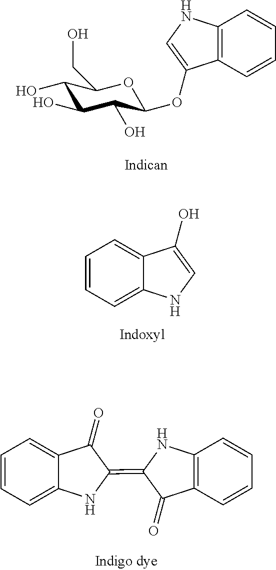

Examples of indican containing plants include Indigofera tinctorial, Indigofera suffruticosa, Indigofera arrecta, Indigofera micheliana, Indigofera coerulea, Indigofera cassioides, Indigofera austalis, Indigofera longiracemosa, and Lonchocarpus cyanescens in the family Fabaceae; Persicaria tinctoria (formerly Polygonum tinctorium) in the family Polygonaceae; and Strobilanthes flaccidifolius, Strobilanthes cusia, and Baphicacanthus cusia in the family Acanthaceae.

The indigo plant population of the present invention has a high indican content. As used herein the term “high indican content” is used to describe plants that produce a higher than average level of indican. In some embodiments, the indigo plant population shows increased indican content relative to a wild or unselected population.

Those skilled in the art will appreciate that the indican content may be assessed with respect to a plurality or even an entire field of plants. An indigo plant may be considered to have high indican content if the high indican content is observed over a plurality of plants (e.g., an average), even if particular individual plants may be lower.

In some embodiments, the population produces an average of at least about 47.83 mg of indican per gram of dry leaf tissue harvested during midseason vegetative growth. In some embodiments, the population produces an average indigo dye yield of at least about 21.24 mg of indigo (C₁₆H₁₀N₂O₂) per gram of dry leaf tissue during midseason vegetative growth and assuming a stoichiometric conversion.

### Genetic Transformation

With the advent of molecular biological techniques that have allowed the isolation and characterization of genes that encode specific protein products, scientists in the field of plant biology developed a strong interest in engineering the genome of plants to contain and express foreign nucleic acids including additional or modified versions of native (endogenous) nucleic acids (optionally driven by a non-native promoter) in order to alter the traits of a plant in a specific manner. Any nucleic acid sequences, whether from a different species, the same species or an artificial sequence, which are introduced into the genome using transformation or various breeding methods, are referred to herein collectively as “transgenes.” Over the last fifteen to twenty years, several methods for producing transgenic plants have been developed, and in particular embodiments the present invention also relates to transformed versions of indigo plants disclosed herein.

Genetic engineering techniques can be used (alone or in combination with breeding methods) to introduce one or more desired added traits into plant, for example, an indigo plant of the population or progeny or plants derived thereof. Once a transgene has been introduction into a plant by genetic transformation, it can be transferred to other plants via conventional breeding.

Plant transformation generally involves the construction of an expression vector that will function in plant cells. Optionally, such a vector comprises one or more nucleic acids comprising a coding sequence for a polypeptide or an untranslated functional RNA under control of, or operatively linked to, a regulatory element (for example, a promoter). In representative embodiments, the vector(s) may be in the form of a plasmid, and can be used alone or in combination with other plasmids, to provide transformed plants using transformation methods as described herein to incorporate transgenes into the genetic material of the plant.

Additional methods include, but are not limited to, expression vectors introduced into plant tissues using a direct nucleic acid transfer method, such as microprojectile-mediated delivery (e.g., with a biolistic device), DNA injection, Agrobacterium-mediated transformation, electroporation, and the like. Transformed plants obtained from the plants (and parts and tissue culture thereof) of the invention are intended to be within the scope of this invention.

### Expression Vectors for Plant Transformation—Selectable Markers

Expression vectors typically include at least one nucleic acid comprising or encoding a selectable marker, operably linked to a regulatory element (for example, a promoter) that allows transformed cells containing the marker to be either recovered by negative selection, e.g., inhibiting growth of cells that do not contain the selectable marker, or by positive selection, e.g., screening for the product encoded by the selectable marker. Many commonly used selectable markers for plant transformation are well known in the transformation art, and include, for example, nucleic acids that code for enzymes that metabolically detoxify a selective chemical agent which may be an antibiotic or an herbicide, or nucleic acids that encode an altered target which is insensitive to the inhibitor. Positive selection methods are also known in the art.

Commonly used selectable markers in plants include, but are not limited to: neomycin phosphotransferase II (nptII) conferring resistance to kanamycin, hygromycin phosphotransferase conferring resistance to the antibiotic hygromycin, bacterial selectable markers that confer resistance to antibiotics (e.g., gentamycin acetyl transferase, streptomycin phosphotransferase, and aminoglycoside-3′-adenyl transferase, selectable markers conferring resistance to herbicides (e.g., glyphosate, glufosinate, or bromoxynil). Selection of transformed plant cells can also be based on screening presumptively transformed plant cells rather than direct genetic selection of transformed cells for resistance to a toxic substance such as an antibiotic; such markers include without limitation alpha-glucuronidase (GUS), alpha-galactosidase, luciferase, and Green Fluorescent Protein (GFP) and mutant GFPs.

### Expression Vectors for Plant Transformation—Promoters

Transgenes included in expression vectors are generally driven by a nucleotide sequence comprising a regulatory element (for example, a promoter). Numerous types of promoters are well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells.

Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in certain tissue are referred to as “tissue-specific.” A “cell type” specific promoter preferentially drives expression in certain cell types in one or more organs, for example, vascular cells in roots or leaves. An “inducible” promoter is a promoter that is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

Many suitable promoters are known in the art and can be selected and used to achieve the desired outcome.

### Signal Sequences for Targeting Proteins to Subcellular Compartments

Transport of polypeptides produced by transgenes to a subcellular compartment such as the chloroplast, vacuole, peroxisome, glyoxysome, cell wall, or mitochondrion, or for secretion into the apoplast, is generally accomplished by means of operably linking a nucleotide sequence encoding a signal sequence to the 5′ and/or 3′ region of a nucleic acid encoding the polypeptide of interest. Signal sequences at the 5′ and/or 3′ end of the coding sequence target the polypeptide to particular subcellular compartments.

The presence of a signal sequence can direct a polypeptide to either an intracellular organelle or subcellular compartment or for secretion to the apoplast. Many signal sequences are known in the art. See, for example, Becker, et al., Plant Mol. Biol., 20:49 (1992); Close, P. S., Master's Thesis, Iowa State University (1993); Knox, C., et al., “Structure and Organization of Two Divergent Alpha-Amylase Genes from Barley,” Plant Mol. Biol., 9:3-17 (1987); Lerner, et al., Plant Physiol., 91:124-129 (1989); Fontes, et al., Plant Cell, 3:483-496 (1991); Matsuoka, et al., PNAS, 88:834 (1991); Gould, et al., J. Cell. Biol., 108:1657 (1989); Creissen, et al., Plant J, 2:129 (1991); Kalderon, et al., A short amino acid sequence able to specify nuclear location, Cell, 39:499-509 (1984); and Steifel, et al., Expression of a maize cell wall hydroxyproline-rich glycoprotein gene in early leaf and root vascular differentiation, Plant Cell, 2:785-793 (1990).

### Foreign Polypeptide Transgenes and Agronomic Transgenes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign polypeptide then can be extracted from a tissue of interest or from total biomass. Protein extraction from plant biomass can be accomplished by known methods which are discussed, for example, by Heney and Orr, Anal. Biochem., 114:92-6 (1981). According to a representative embodiment, the transgenic plant provided for commercial production of foreign protein is a plant of the invention. In another embodiment, the biomass of interest is seed and/or fruit.

Likewise, by means of the present invention, agronomic transgenes and other desired added traits can be expressed in transformed plants (and their progeny, e.g., produced by breeding methods). More particularly, plants can be genetically engineered to express various phenotypes of agronomic interest or other desired added traits. Exemplary nucleic acids of interest in this regard conferring a desired added trait(s) include, but are not limited to, those transgenes that confer resistance to confer resistance to plant pests (e.g., nematode or insect) or disease (e.g., fungal, bacterial or viral), transgenes that confer herbicide tolerance, transgenes that confer male sterility.

In embodiments, the transgene encodes a non-translated RNA (e.g., RNAi) that is expressed to produce targeted inhibition of gene expression, thereby conferring the desired trait on the plant.

In embodiments, the transgene encodes the machinery used for gene editing techniques.

Any transgene, including those exemplified above, can be introduced into the plants of the invention through a variety of means including, but not limited to, transformation (e.g., genetic engineering techniques), conventional breeding, and introgression methods to introduce the transgene into other genetic backgrounds.

### Methods for Plant Transformation

Numerous methods for plant transformation have been developed, including biological and physical plant transformation protocols. See, for example, Miki, et al., “Procedures for Introducing Foreign DNA into Plants” in Methods in Plant Molecular Biology and Biotechnology, Glick and Thompson Eds., CRC Press, Inc., Boca Raton, pp. 67-88 (1993). In addition, expression vectors and in vitro culture methods for plant cell or tissue transformation and regeneration of plants are available. See, for example, Gruber, et al., “Vectors for Plant Transformation” in Methods in Plant Molecular Biology and Biotechnology, Glick and Thompson Eds., CRC Press, Inc., Boca Raton, pp. 89-119 (1993). Commonly used plant transformation methods include agrobacterium-mediated transformation and direct transgene transfer methods (e.g., microprojectile-mediated transformation, sonication, liposome or spheroplast fusion, and electroporation of protoplasts or whole cells).

Following transformation of plant target tissues, expression of selectable marker transgenes (e.g., as described above) allows for preferential selection of transformed cells, tissues and/or plants, using regeneration and selection methods now well known in the art.

The foregoing methods for transformation are typically used to produce a transgenic line. The transgenic line can then be crossed with another (non-transgenic or transgenic) line in order to produce a new transgenic line. Alternatively, a transgene that has been engineered into a particular plant using transformation techniques can be introduced into another plant or line using traditional breeding (e.g., backcrossing) techniques that are well known in the plant breeding arts. For example, a backcrossing approach can be used to move an engineered transgene from a public, non-elite inbred line into an elite inbred line, or from an inbred line containing a foreign transgene in its genome into an inbred line or lines which do not contain that transgene. As used herein, “crossing” can refer to a simple X by Y cross, or the process of backcrossing, depending on the context.

### Targeting Induced Local Lesions in Genomes (TILLING)

Breeding schemes of the present application can include crosses with TILLING® plant cultivars. TILLING® is a method in molecular biology that allows directed identification of mutations in a specific gene. TILLING® was introduced in 2000, using the model plant Arabidopsis thaliana. TILLING® has since been used as a reverse genetics' method in other organisms such as zebrafish, corn, wheat, rice, soybean, tomato and lettuce.

The method combines a standard and efficient technique of mutagenesis with a chemical mutagen (e.g., Ethyl methanesulfonate (EMS)) with a sensitive DNA screening-technique that identifies single base mutations (also called point mutations) in a target gene. EcoTILLING is a method that uses TILLING® techniques to look for natural mutations in individuals, usually for population genetics analysis (see Comai, et al., 2003 The Plant Journal 37, 778-786; Gilchrist et al. 2006 Mol. Ecol. 15, 1367-1378; Mejlhede et al. 2006 Plant Breeding 125, 461-467; Nieto et al. 2007 BMC Plant Biology 7, 34-42, each of which is incorporated by reference hereby for all purposes). DEcoTILLING is a modification of TILLING® and EcoTILLING which uses an inexpensive method to identify fragments (Garvin et al., 2007, DEco-TILLING: An inexpensive method for SNP discovery that reduces ascertainment bias. Molecular Ecology Notes 7, 735-746).

The TILLING® method relies on the formation of heteroduplexes that are formed when multiple alleles (which could be from a heterozygote or a pool of multiple homozygotes and heterozygotes) are amplified in a PCR, heated, and then slowly cooled. A “bubble” forms at the mismatch of the two DNA strands (the induced mutation in TILLING® or the natural mutation or SNP in EcoTILLING), which is then cleaved by single stranded nucleases. The products are then separated by size on several different platforms.

Several TILLING® centers exists over the world that focus on agriculturally important species: UC Davis (USA), focusing on Rice; Purdue University (USA), focusing on Maize; University of British Columbia (CA), focusing on Brassica napus; John Innes Centre (UK), focusing on Brassica rapa; Fred Hutchinson Cancer Research, focusing on Arabidopsis; Southern Illinois University (USA), focusing on Soybean; John Innes Centre (UK), focusing on Lotus and Medicago; and INRA (France), focusing on Pea and Tomato. More detailed description on methods and compositions on TILLING® can be found in U.S. Pat. No. 5,994,075, US 2004/0053236 A1, WO 2005/055704, and WO 2005/048692, each of which is hereby incorporated by reference for all purposes.

Thus, in some embodiments, the breeding methods of the present disclosure include breeding with one or more TILLING plant lines with one or more identified mutations.

### Additional Methods for Genome Engineering of Indigo

In general, methods to transform, modify, edit or alter plant endogenous genomic DNA include altering the plant native DNA sequence or a pre-existing transgenic sequence including regulatory elements, coding and non-coding sequences. These methods can be used, for example, to target nucleic acids to pre-engineered target recognition sequences in the genome. Such pre-engineered target sequences may be introduced by genome editing or modification. As an example, a genetically modified plant variety is generated using “custom” or engineered endonucleases such as meganucleases produced to modify plant genomes (see e.g., WO 2009/114321; Gao et al. (2010) Plant Journal 1:176-187). Another site-directed engineering method is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system and other similar methods. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1, incorporated herein by reference).

### Locus Conversion

When the term “plant” is used in the context of the present invention, this term also includes any locus conversions of that plant or variety. The term “locus converted plant” as used herein refers to those plants that are developed, for example, by backcrossing, genome editing, genetic transformation and/or mutation, wherein essentially all of the desired morphological and physiological characteristics are recovered in addition to the one or more genes introduced into the variety. To illustrate, backcrossing methods can be used with the present invention to improve or introduce a characteristic into the variety. The term “backcrossing” as used herein refers to the repeated crossing of a hybrid progeny back to the recurrent parent, e.g., backcrossing 1, 2, 3, 4, 5, 6, 7, 8, 9, or more times to the recurrent parent. The parental plant that contributes the gene for the desired characteristic is termed the “nonrecurrent” or “donor parent.” This terminology refers to the fact that the nonrecurrent parent is generally used one time in the breeding e.g., backcross) protocol and therefore does not recur. The gene that is transferred can be a native gene, a mutated native gene or a transgene introduced by genetic engineering techniques into the plant (or ancestor thereof). The parental plant into which the gene(s) from the nonrecurrent parent are transferred is known as the “recurrent” parent as it is used for multiple rounds in the backcrossing protocol. Poehlman & Sleper (1994) and Fehr (1993). In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a plant is obtained wherein essentially all of the desired morphological and physiological characteristics of the recurrent parent are recovered in the converted plant in addition to the transferred gene(s) and associated trait(s) from the nonrecurrent parent.

### Tissue Culture

Further reproduction of indigo plants can occur by tissue culture and regeneration. Tissue culture of various tissues and regeneration of plants therefrom is well known and widely published. For example, reference may be had to Teng, et al., HortScience, 27:9, 1030-1032 (1992); Teng, et al., HortScience, 28:6, 669-1671 (1993); Zhang, et al., Journal of Genetics and Breeding, 46:3, 287-290 (1992); Webb, et al., Plant Cell Tissue and Organ Culture, 38:1, 77-79 (1994); Curtis, et al., Journal of Experimental Botany, 45:279, 1441-1449 (1994); Nagata, et al., Journal for the American Society for Horticultural Science, 125:6, 669-672 (2000); and Ibrahim, et al., Plant Cell Tissue and Organ Culture, 28(2), 139-145 (1992). It is clear from the literature that the state of the art is such that these methods of obtaining plants are routinely used and have a very high rate of success. Thus, another aspect of this invention is to provide cells which upon growth and differentiation produce indigo plants having desired characteristics of the indigo plant population. Optionally, indigo plants can be regenerated from the tissue culture of the invention comprising all or essentially all of the physiological and morphological characteristics of the indigo plant population.

As used herein, the term “tissue culture” indicates a composition comprising isolated cells of the same or a different type or a collection of such cells organized into parts of a plant. Exemplary types of tissue cultures are protoplasts, calli, meristematic cells, and plant cells that can generate tissue culture that are intact in plants or parts of plants, such as leaves, pollen, embryos, roots, root tips, anthers, pistils, flowers, seeds, petioles, suckers, and the like. Means for preparing and maintaining plant tissue culture are well known in the art. By way of example, a tissue culture comprising organs has been used to produce regenerated plants. U.S. Pat. Nos. 5,959,185, 5,973,234, and 5,977,445 describe certain techniques.

### Additional Breeding Methods

This invention is also directed to methods for producing an indigo plant by crossing a first parent indigo plant with a second parent indigo plant wherein the first or second parent indigo plant is an indigo plant of the population. Further, both first and second parent indigo can come from the indigo plants of the population. Thus, any of the following exemplary methods the indigo plants of the population are part of this invention: selfing, backcrosses, hybrid production, crosses to populations, double haploid production, and the like. All plants produced using an indigo plant of the population as at least one parent are within the scope of this invention, including those developed from indigo plants derived from the indigo plants of the population. Advantageously, an indigo plant of the population can be used in crosses with other, different, indigo plants to produce the first generation (F₁) indigo hybrid seeds and plants with desirable characteristics. The indigo plants of the invention can also be used for transformation where exogenous transgenes are introduced and expressed by the plants of the invention. Genetic variants created either through traditional breeding methods or through transformation of the cultivars of the invention by any of a number of protocols known to those of skill in the art are intended to be within the scope of this invention.

The following describes exemplary breeding methods that may be used with the indigo plant population in the development of further indigo plants. One such embodiment is a method for developing progeny indigo plants in an indigo plant breeding program comprising: obtaining a plant, or a part thereof, of the indigo plants of the population, utilizing said plant or plant part as a source of breeding material, and selecting a progeny plant with molecular markers in common with an indigo plant of the population and/or with some, all or essentially all of the morphological and/or physiological characteristics of the indigo plant population. In representative embodiments, the progeny plant has at least 1, 2, 3, 4, 5, 6, 7, 8, 9, 10 or more of the morphological and physiological characteristics of the indigo plants of the population, or even all of the morphological and physiological characteristics of the indigo plant population so that said progeny indigo plant is not significantly different for said traits than the indigo plants of the population, as determined at the 5% significance level when grown in the same environmental conditions; optionally, with the presence of one or more desired additional traits (e.g., male sterility, disease resistance, pest or insect resistance, herbicide resistance, and the like). Breeding steps that may be used in the breeding program include pedigree breeding, backcrossing, mutation breeding and/or recurrent selection. In conjunction with these steps, techniques such as RFLP-enhanced selection, genetic marker enhanced selection (for example, SSR markers) and/or and the making of double haploids may be utilized.

Another representative method involves producing a population of progeny plants, comprising crossing an indigo plant of the population with another indigo plant, thereby producing a population of indigo plants that, on average, derives at least 6.25%, 12.5%, 25%, 30%, 35%, 40%, 45%, 50%, 55%, 60%, 65%, 70%, 80%, 85%, 90%, 95%, 96%, 97%, 98% or 99% of its alleles from the indigo plants of the population, e.g., at least about 6.25%, 12.5%, 25%, 30%, 35%, 40%, 45%, 50%, 55%, 60%, 65%, 70%, 80%, 85%, 90%, 95%, 96%, 97%, 98% or 99% of the genetic complement of the indigo plants of the population. One embodiment of this invention is the indigo plant produced by this method and that has obtained at least 6.25%, 12.5%, 25%, 30%, 35%, 40%, 45%, 50%, 55%, 60%, 65%, 70%, 80%, 85%, 90%, 95%, 96%, 97%, 98% or 99% of its alleles from the indigo plants of the population, and optionally is the result of a breeding process comprising one or two breeding crosses and one or more of selfing, sibbing, backcrossing and/or double haploid techniques in any combination and any order. In embodiments, the breeding process does not include a breeding cross, and comprises selfing, sibbing, backcrossing and or double haploid technology. A plant of this population may be selected and repeatedly selfed or sibbed with an indigo plant resulting from these successive filial generations. Another approach is to make double haploid plants to achieve homozygosity.

One of ordinary skill in the art of plant breeding would know how to evaluate the traits of two plant varieties to determine if there is no significant difference between the two traits expressed by those varieties. For example, see Fehr and Walt, Principles of Cultivar Development, pp. 261-286 (1987). In embodiments, the invention encompasses progeny indigo plants having a combination of at least 2, 3, 4, 5, 6, 7, 8, 9, 10 or more of the characteristics as described herein for the indigo plants of the population, so that said progeny indigo plant is not significantly different for said traits than the indigo plants of the population, as determined at the 5% significance level when grown in the same environmental conditions. Using techniques described herein and those known in the art, molecular markers may be used to identify said progeny plant as progeny of the indigo plants of the population. Mean trait values may be used to determine whether trait differences are significant, and optionally the traits are measured on plants grown under the same environmental conditions.

Progeny of the indigo plants of the population may also be characterized through their filial relationship with the indigo plants of the population, as for example, being within a certain number of breeding crosses of the indigo plants of the population. A breeding cross is a cross made to introduce new genetics into the progeny, and is distinguished from a cross, such as a self or a sib cross or a backcross to an indigo plant of the population as a recurrent parent, made to select among existing genetic alleles. The lower the number of breeding crosses in the pedigree, the closer the relationship between the indigo plants of the population and its progeny. For example, progeny produced by the methods described herein may be within 1, 2, 3, 4, 5 or more breeding crosses of the indigo plants of the population.

In representative embodiments, an indigo plant derived from the indigo plants of the population comprises cells comprising at least one set of chromosomes derived from the indigo plants of the population. In embodiments, the indigo plant or population of indigo plants derived from the indigo plants of the population comprises, on average, at least 6.25%, 12.5%, 25%, 30%, 35%, 40%, 45%, 50%, 55%, 60%, 65%, 70%, 80%, 85%, 90%, 95%, 96%, 97%, 98% or 99% of its alleles from the indigo plants of the population, e.g., at least about 6.25%, 12.5%, 25%, 30%, 35%, 40%, 45%, 50%, 55%, 60%, 65%, 70%, 80%, 85%, 90%, 95%, 96%, 97%, 98% or 99% of the genetic complement of the indigo plants of the population, and optionally is the result of a breeding process comprising one or two breeding crosses and one or more of selfing, sibbing, backcrossing and/or double haploid techniques in any combination and any order. In embodiments, the breeding process does not include a breeding cross, and comprises selfing, sibbing, backcrossing and or double haploid technology. In embodiments, the indigo plant derived from the indigo plants of the population is one, two, three, four, five or more breeding crosses removed from the indigo plants of the population.

In representative embodiments, a plant derived from the indigo plants of the population is a double haploid plant, a hybrid plant or an inbred plant.

In embodiments, a hybrid or derived plant from the indigo plants of the population comprises a desired added trait. In representative embodiments, an indigo plant derived from the indigo plants of the population comprises all of the morphological and physiological characteristics of the population (e.g., high indican content). In embodiments, the indigo plant derived from the indigo plants of the population comprises essentially all of the morphological and physiological characteristics of the population (e.g., high indican content), with the addition of a desired added trait.

Those skilled in the art will appreciate that any of the traits described above with respect to plant transformation methods can be introduced into a plant of the invention (e.g., the indigo plants of the population and hybrid indigo plants and other indigo plants derived therefrom) using breeding techniques.

The foregoing disclosure has been described in detail by way of illustration and example for purposes of clarity and understanding. However, it will be obvious that certain changes and modifications such as single gene modifications and mutations, somoclonal variants, variant individuals selected from large populations of the plants and the like may be practiced within the scope of the disclosure, as limited only by the scope of the appended claims.

### EXAMPLES

Stony Creek began breeding (selecting for) its I. suffruticosa crop in 2016 and in 2019 began to screen large numbers of individual plants from selected populations using a proprietary indican assay screen. Upon completion of the population assay, these “breeding nurseries” were culled of the lowest performing individuals (only retaining the top 15%) and allowed to mass-cross through open pollination of the high-performing individuals.

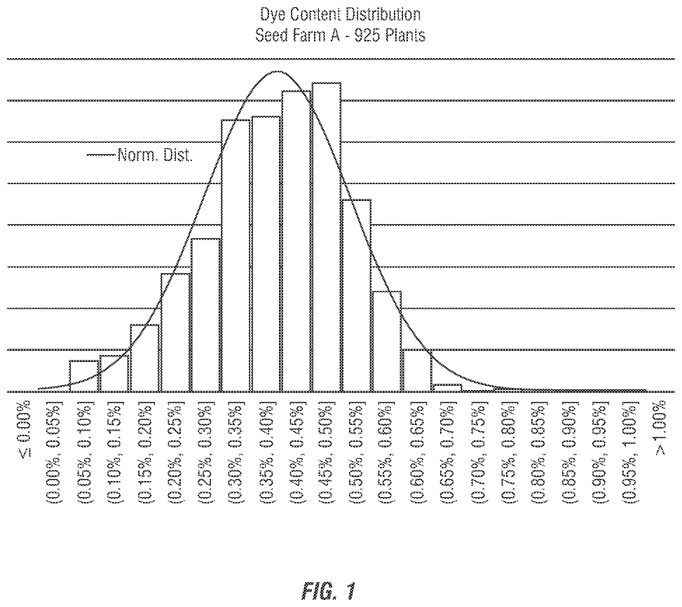

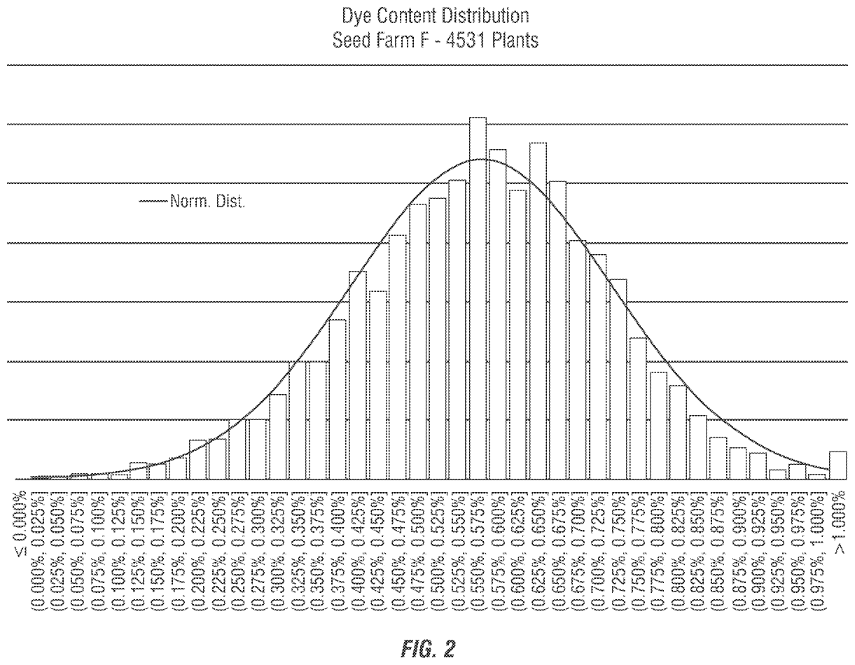

In 2019, two fields were used as breeding nurseries—one in TN with roughly 1,000 plants (FIG. 1), and one in South FL with roughly 4,500 plants (FIG. 2). Indican appears to be a function of both genetics and environmental conditions, so “indican content” or “potential dye yield” needs to be measured relative to unimproved crop grown under similar conditions, but it should be apparent that the distribution of indican per leaf mass amongst non-culled breeding nurseries had a near Gaussian distribution (stretching to 0 indican content) indicating an unimproved or near-wild population.

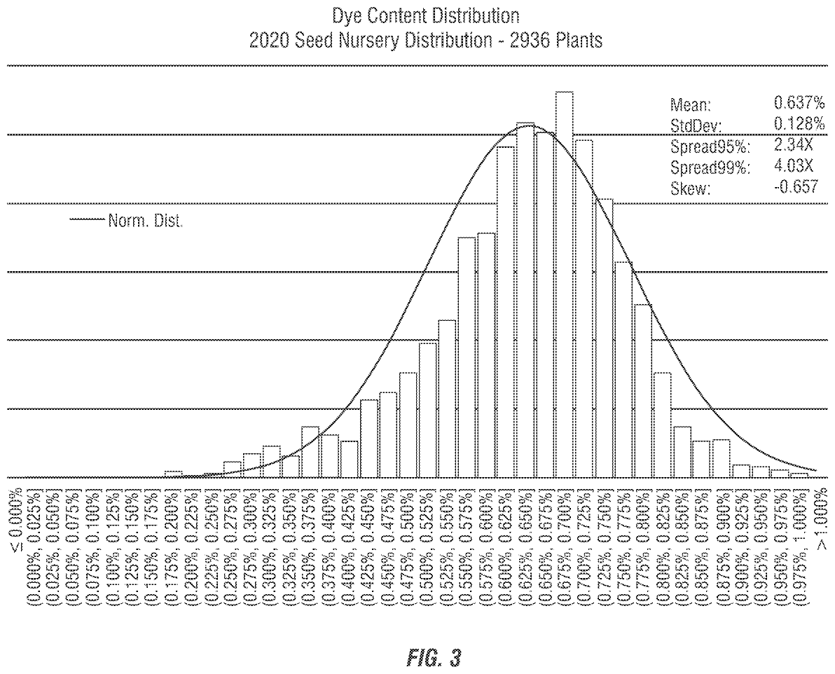

Seed collected from these 2019 breeding nurseries was used for producing the 2020 breeding nursery located in TN (same climate and seasonal timing as the 2019 TN breeding nursery). FIG. 3 illustrates the indican content per leaf mass prior to selection and culling. It should be apparent that not only has the distribution tightened and shifted considerably up the axis, but that the distribution has a significant negative skew (−0.657) vs the normal distribution, suggesting the retention of some lower performing genetics.

### Deposits

Applicant(s) will make a deposit of at least 625 seeds of the Indigofera suffruticosa population with the American Type Culture Collection (ATCC), Manassas, Va. 20110 USA, ATCC Deposit No. ______. The seeds deposited with the ATCC on ______ will be taken from the deposit maintained by Stony Creek Colors, 921 Central Ave W, Springfield, Tenn. 37172 since prior to the filing date of this application. Access to this deposit will be available during the pendency of the application to the Commissioner of Patents and Trademarks and persons determined by the Commissioner to be entitled thereto upon request. Upon issue of claims, the Applicant(s) will make available to the public, pursuant to 37 CFR 1.808, a deposit of at least 2500 seeds of the Indigofera suffruticosa population with the American type Culture Collection (ATCC), 10801 University Boulevard, Manassas, Va. 20110-2209. This deposit of the Indigofera suffruticosa population will be maintained in the ATCC depository, which is a public depository, for a period of 30 years, or 5 years after the most recent request, or for the enforceable life of the patent, whichever is longer, and will be replaced if it becomes nonviable during that period. Additionally, Applicants have or will satisfy all the requirements of 37 C.F.R. §§ 1.801-1.809, including providing an indication of the viability of the sample. Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

What is claimed is:

1. An assemblage of indigo seeds of a population with high indican content, a representative sample having been deposited under ATCC Accession No. PTA-XXXX.

2. A population of indigo plants, or plant parts thereof, produced by growing the seeds of claim 1.

3. The population of indigo plants of claim 2, wherein the population produces a higher than average level of indican.

4. The population of indigo plants of claim 2, wherein the population produces an average of at least about 47 mg of indican per gram of dry leaf tissue harvested during midseason vegetative growth.

5. The population of indigo plants of claim 2, wherein the population produces an average indigo dye yield of at least about 21 mg of indigo per gram of dry leaf tissue during midseason vegetative growth assuming a stoichiometric conversion.

6. The population of indigo plants of claim 2, wherein the plant parts are leaves, ovules, pollen, or cells.

7. The population of indigo plants of claim 6, wherein the plant parts are leaves.

8. A method of producing a population of progeny indigo plants with higher than average indican content, the method comprising:
selecting an indigo plant with high indican content from the population of indigo plants of claim 2;
crossing the selected indigo plant with itself or a second indigo plant;
harvesting the resulting seeds; and
growing a population of progeny indigo plants from the seeds.

9. A population of progeny indigo plants produced by the method of claim 8.

10. The population of progeny indigo plants of claim 9, wherein the population produces a higher than average level of indican.

11. A method of increasing indigo dye yield per area of land, the method comprising:
planting the indigo seeds of claim 1;
allowing the seeds to grow into a population of indigo plants;
harvesting the population of indigo plants, or plant parts thereof; and
obtaining indigo dye from the harvested indigo plants, or the plant parts thereof.

12. The method of claim 11, wherein the plant parts are leaves.

13. The method of claim 11, wherein the indigo dye yield per area of land is increased relative to an unimproved wild population.

14. A method for producing a processed product from a population of indigo plants having high indican content, the method comprising:
growing a population of indigo plants from the indigo seeds of claim 1;
harvesting the plants, or plant parts thereof; and
processing the plants, or the plant parts thereof, to produce a processed product.

15. The method of claim 14, wherein the processed product comprises indican or indigo dye.

16. The method of claim 14, wherein the processing comprises extracting, purifying, and/or isolating.

17. The method of claim 14, wherein the parts are leaves.

18. A method for developing an indigo variety in a plant breeding program, comprising applying plant breeding techniques comprising recurrent selection, backcrossing, pedigree breeding, marker enhanced selection, mutation breeding, genome editing, or genetic modification to the population of indigo plants of claim 2 to develop an indigo variety with high indican content.

19. A method of developing a new indigo plant or cultivar with higher-than-average indican content comprising:
obtaining seed from the assemblage of seed of claim 1;
allowing said seed to self-pollinate to form progeny seed
selecting from said progeny seed with higher than average indigo content, wherein said indigo content is higher than the assemblage of seed of claim 1.

20. A method of introgressing higher than average indican content into an indica cultivar comprising
using the seeds of claim 1 as a source of breeding material and
backcrossing plants grown from said from said source to another second indica cultivar for a sufficient number of times so that the cultivar is regenerated with the trait of higher than average indican content introgressed therein.
